(19) United States
(12) Patent Application Publication
(10) Pub. No.: US 2017/0143915 A1
(43) Pub. Date: May 25, 2017

(54) AEROSOLISATION ENGINE FOR LIQUID DRUG DELIVERY BACKGROUND

(71) Applicant: The Technology Partnership Plc, Royston (GB)

(72) Inventors: Daniel Geoffrey Tyler Strange, Cambridge (GB); Robert Gordon Maurice Selby, Melbourn (GB); Romain Ulysses Gabriel Guion, Cambridge (GB); Timothy James Phillips, Cambridge (GB); William Richardson, Royston (GB)

(73) Assignee: The Technology Partnership Plc, Royston (GB)

(21) Appl. No.: 15/310,346

(22) PCT Filed: May 13, 2015
(86) PCT No.: PCT/GB2015/051413

§ 371 Date: Nov. 10, 2016

(30) Foreign Application Priority Data
May 14, 2014 (GB) 1408561.7

(51) Int. Cl. A61M 11/00; A61F 9/00; B05B 1/26; A61M 15/00; A61M 15/08

(52) U.S. Cl. CPC A61M 11/007; A61M 11/002; A61M 11/003; A61M 15/0021; A61M 15/08; B05B 1/262; A61F 9/0008; A61M 2205/0238; A61M 2205/8225; A61M 2205/8281; A61M 2210/0612

(57) ABSTRACT

A spray device for generating an aerosol of a liquid such as a medicament. The device includes a perforate element comprising one or more nozzles, each nozzle having an inlet and an outlet. A drive mechanism causes, in use, liquid to be driven through the one or more nozzles, thereby forming a liquid spray having one or more streams of liquid. At least one impaction surface is provided onto which, in use, the liquid impacts, the impaction surface being located downstream of the nozzle outlet(s).

Aerosols are highly effective and user-friendly methods of delivering pharmaceutical ingredients to the lungs, nose, and eyes. Delivery is targeted, with fast uptake. Aerosols are also simple for users to apply without direct user contact with tissue, avoiding many of the complications from applying topical medicines such as eye drops.

Key attributes of aerosol performance are droplet size distribution, plume velocity, plume duration, and plume angle. The precise combination of attributes depends on the delivery target and active pharmaceutical ingredient. In inhalation, droplets larger than 5.8 μm will not effectively reach the deep lung but will instead deposit in the upper bronchials and throat. Plumes with velocities greater than 10 m/s, as is typical for pressurised metered dose inhalers, will deposit substantially more drug on the throat than “soft mist” inhalers where the plume velocity is on the order of 1 m/s. The long plume durations of soft mist inhalers may also assist with correct user technique and coordination, encouraging users to breathe in slowly, rather than with short sharp breaths.

In nasal delivery, droplets are not intended to be inhaled and should be larger than 10 μm. However, droplets much larger than 30 μm will typically agglomerate and drip out of the nose. Nasal sprays with a wide spray angle are more likely to deposit in the anterior region of the nose rather than the turbinate region. Furthermore, unlike inhalers, the droplets must have sufficient forward momentum to navigate to the turbinate region of the nose, without the user breathing in.

There are a wide range of methods for generating aerosols with small droplets. However, typically it is difficult to decouple the parameters influencing droplet diameter with those determining plume velocity, duration and geometry. Regularly sized droplets can be formed by passing fluid streams through a small nozzle. The stream will naturally breakup due to the growth of unstable environmental perturbations that act to reduce the surface energy of the stream (the Plateau-Rayleigh instability). The droplets will tend to have a diameter that is related to the most unstable wavelength, which itself is a function of the fluid stream radius. However the fluid stream must have sufficient velocity for the stream to escape the nozzle as a continuous jet, without wetting the front face, otherwise larger droplets will be produced. Hence small droplets can be produced but only at relatively high velocities with a long breakup length.

U.S. Pat. No. 5,472,143 discloses methods of generating plumes of fine droplets by colliding high velocity jets together. The resulting jet has a low forward momentum, which can be tailored by the angle of the colliding jets. However, in order to achieve long plume durations with small quantities of drug, the flow rate of the stream passing through the nozzle must be very small (10 μl/s). Consequently, the nozzles must also have a small nozzle diameter (<10 μm). It is expensive to manufacture nozzles for this purpose as they must be very well aligned to ensure the jets collide. A typical silicon microfluidics chip which could be used for this purpose costs on the order of 0.5 GBP.

Hence there is a need for a low cost method of generating low-speed mists of aerosols with droplets sizes from 2.5 μm to 30 μm, using a handheld portable device, with near independent control of droplet size, plume velocity, plume duration, and geometry.

Many air-blast nebulisers and similar portable devices such as those disclosed in JP 02-116379 and US20130228176 produce a fine-mist by colliding coarser droplets into a baffle to cause secondary breakup of droplets. The outward plume velocity is relatively independent of the initial jet velocity, due to the deceleration during impact. However, these devices have relatively wide droplet distributions due to the distribution of coarse droplets, which themselves are produced by stochastic air blast atomisation, impaction at low droplet speeds, or other methods [Finlay, W. H., The Mechanics of Inhaled Pharmaceutical Aerosols, An Introduction. Academic Press, London, 2001]. In nebulisers, further baffles may be used to filter out the droplets and then recycle the fluid. This is not practicable for a non-continuous portable device such as an inhaler. It is advantageous to control the governing parameters of the fluid prior to impact and hence tightly control the parameters influencing the final droplet distribution. This can be done by forcing a liquid through a precision nozzle at high pressure, such that the jet diameter is determined by the nozzle hole diameter and the jet speed is determined by the pressure.

Splash plate nozzles, such as that disclosed by U.S. Pat. No. 5,762,005, are a well-known method of aerosolising industrial fluids into coarse droplet sprays (droplets in the region of 400 μm as defined by the American Society of Agricutural and Biological Engineers in classification system ASABE S-572.1), whereby liquid is forced through a nozzle at high pressure and impacted on a splash plate, before the jet breaks up. They are typically used for applications that require a large flow rate (fire sprinklers) or where a viscous fluid is used (black liquor nozzles in recovery boilers) [Sarchami, A and Ashgriz N, “Splash Plate Atomizers” in N. Ashgriz (ed.) Handbook of Atomization and Sprays, Springer, New York, 2011]. To achieve the large flow rates and/or ejection of viscous fluids, fluid is forced through a large wide nozzle (approximately 1 mm diameter). The fluid is collided with a flat splash plate, which has an angle of 35-55 degrees to the jet. After impact, the jet forms a film on the plate and then breaks up into regularly sized droplets. Similarly, pin impaction nozzles are commonly used for generating water fogs of droplets, particularly for humidification of industrial gas turbines. In such arrangements water is forced through an orifice 125 to 400 μm in diameter at pressures in excess of 25 bar, to impact a pin that is substantially the same size as the orifice.

Both splash plate nozzles and pin impaction nozzles are advantageous as the large contact area between the fluid and air that is achieved after impact results in efficient atomisation. Furthermore the speed and size of the resultant droplets is not directly related to the size of the nozzle; large nozzle size to droplet size ratios can be achieved. However, there are a number of different mechanisms that can contribute to droplet breakup, depending on the relative proportions of kinetic energy of the jet, surface energy and viscous dissipation on impact [Ahmed, M., Ashgriz, N., and Tran, H. N., “Influence of Breakup Regimes on the Droplet Size Produced by Splash-Plate Nozzles”, AIAA Journal, Vol. 47, No. 3, 2009 p516-522]. It is not well understood how liquid forced through a much smaller nozzle will behave when impacted onto a plate many times larger than the nozzle-size, and whether this will result in a relatively mono-disperse fine mist of respirable droplets.

Furthermore, the flow rates and dose volumes that are desired for medical therapies are orders of magnitude smaller than those typically achieved with splash plate nozzles. Consequently, it is possible to achieve much higher jet velocities (>100 m/s), than what is achieved with splash nozzles (typically 30 m/s or less), even with a portable device. The energy required to accelerate a typical dose volume of drug (10-100 μl) to speeds of 100 m/s is on the order of 0.5 J and can be provided by a low cost energy storage mechanism such as spring. This is advantageous as higher jet velocities result in greater reductions in droplet size after impact, due to the larger ratio of kinetic energy to surface tension in the jet. High speed jets are also less sensitive to variations in surface tension near the nozzle and hence performance is likely to be more consistent.

Lastly, splash plate nozzles are typically used to produce coarse droplet sprays—these are not strongly affected by the airflow surrounding the plume. In contrast, the speed and direction of fine or very fine droplets with diameters of 30 μm or less (as would be desired for medical therapies) is strongly affected by the airflow surrounding the plume. The 100 m/s jet ejecting from a nozzle will accelerate the air surround it. Even after the jet impacts the baffle, the annulus of air surrounding the jet will continue to flow past the baffle entraining droplets produced by the impact. Hence, when fine droplets are produced, as is likely with jets emanating from holes at diameters of less than 100 μm, an impact surface external to the nozzle outlet can be used to control and direct the velocity and direction of the plume by modifying the airflow generated by the jet. This is in contrast to methods where a collision surface is integrated into the nozzle such as the method disclosed in U.S. Pat. No. 5,472,143, where there is little to no possibility for controlling the airflow. Engineering plume speed and shape is of critical interest for aerosolised drug delivery.

### SUMMARY OF THE INVENTION

The present invention provides a spray device for generating an aerosol of a liquid medicament such as a liquid drug, solution, suspension or colloid, the device including a perforate element comprising one or more nozzles, each nozzle having an inlet and an outlet, a drive mechanism for causing, in use, liquid to be driven through the one or more nozzles, thereby forming a liquid spray having one or more streams of liquid and at least one impaction surface onto which, in use, the liquid impacts, the impaction surface being located downstream of the nozzle outlet(s).

The present invention also provides a spray device for generating an aerosol, the device including a perforate element comprising one or more nozzles, each nozzle having an inlet and an outlet, a drive mechanism for causing, in use, liquid to be driven through the one or more nozzles, thereby forming a liquid spray having one or more streams of liquid, and at least one baffle having an impaction surface onto which, in use, the liquid impacts, the impaction surface being located downstream of the nozzle outlet(s).

The present invention also provides a method of generating an aerosol of a liquid medicament such as a liquid drug, solution, suspension or colloid, the method comprising the steps of providing a liquid to an inlet side of a perforate element having one or more nozzles, driving the liquid through the perforate element to create a liquid spray having one or more streams of liquid, and impacting the liquid spray onto an impaction surface located downstream of the nozzle outlet(s) to create an aerosol.

Pressures in excess of 10 bar (likely 100 bar) are typically applied to the fluid, forcing it through the exit nozzles at velocities in excess of 30 m/s (typically 100 m/s). The high velocity jet or jets collide with the impinging surface, breaking up into droplets with controllable mean droplet diameters (DV50) preferably as low as 2.5 μm or as large as 30 μm. The direction and velocity of the resultant plume cloud is strongly affected both by the angle and shape of the impacting surface, and by the velocity of the air external to the nozzle.

The nozzle holes may have a diameter less than 100 μm, though typically in the range of 2-70 μm. The larger the holes the greater the flow rate of the liquid through the precision mesh. The nozzles may be manufactured by laser drilling (preferred), by electroforming, or perhaps even moulding for large holes. A second precision mesh, with many (typically 1000) holes that are slightly smaller than the nozzle hole diameters may be placed directly upstream of the nozzle mesh, to act as a filter. The filter can be manufactured using the same manufacturing methods, amongst others.

The impingement surface is located external to the nozzle plate, but close enough such that the jet does not fully breakup into droplets before impacting the surface. It has four functions: it should provide a surface with which the fluid jet collides and breakups into regularly sized droplets; it should minimise the amount of fluid remaining on the surface; it should reduce the kinetic energy of the droplets and cause them to breakup in a desired direction; finally, it should direct the airflow entrained by the fluid jet around itself, affecting the resultant direction and velocity of the plume.

The impingement surface can consist of a wide flat plate though this will halt the velocity of the droplets and impede the droplet cloud from travelling around the plate. An angled baffle will allow the droplets produced after impact to retain some forward momentum. A thin plate or blade that presents a minimal cross-sectional area will substantially reduce the forward momentum of the droplets, but will not significantly impede the air flow round the baffle.

The impinging surface may be placed inside a component such as a mouthpiece, nosepiece or similar user interface. It may even be an integral part of the user interface, such as an angled surface. Air inlets may be placed upstream of the impaction surface or similar to ensure that air is drawn in behind the impaction surface, entraining droplets that are produced as a result of the collision. The shape of the component may also be designed with a converging or diverging outlet, to ensure that the air stream from the air inlets to the outlet travels behind the baffle, and to affect the plume velocity.

The pressure can be provided to the device by a piston with a diameter typically 4 mm or less, which is driven by a helical spring. Alternatively, the pressure could be applied by a compressed air or gas source.

The proposed invention provides significant control over the plume generated by the process. The droplet size distribution produced is strongly dependent on the pressure applied to the fluid, but only weakly correlated with the nozzle diameter. The flow rate and hence plume duration can then be adjusted independently by appropriate selection of the hole diameter and number of holes. Finally the plume velocity and shape can be controlled by appropriate design of the baffle and user interface.

### DETAILED DESCRIPTION

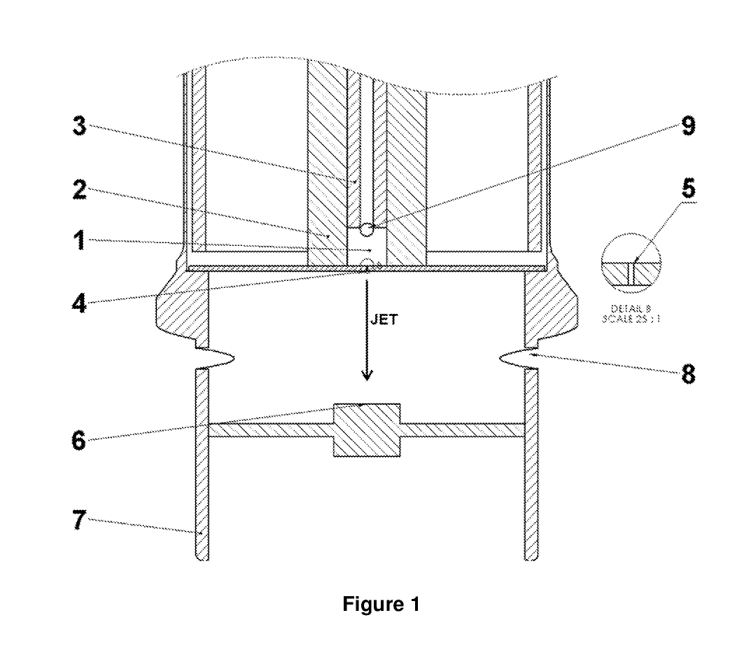

FIG. 1 is a side cross-sectional view of a device according to the present invention.

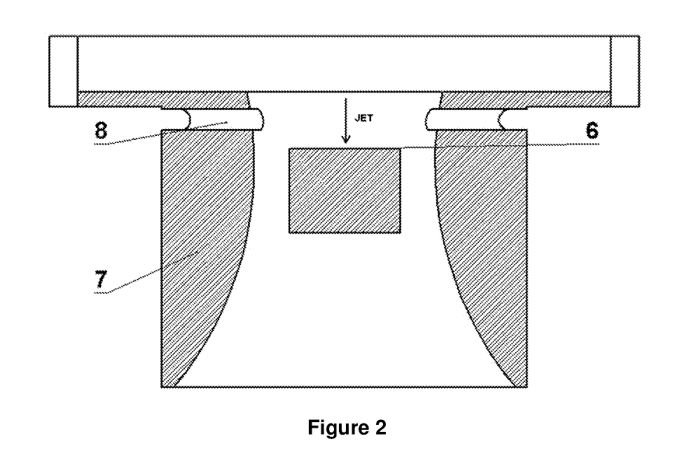

FIG. 2 is a side cross-sectional view of a user-interface with air inlets upstream of the impaction surface and a constriction near the impaction surface.

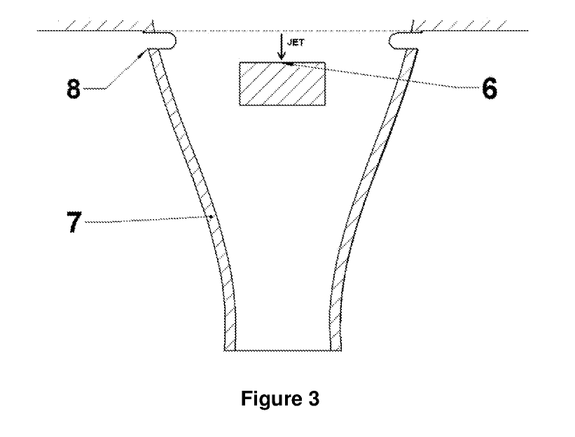

FIG. 3 is a side cross-sectional view of a user-interface with a flat baffle.

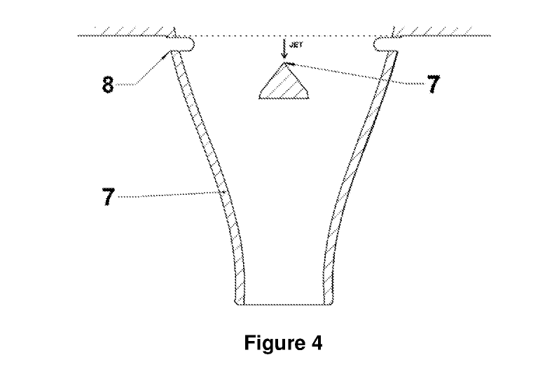

FIG. 4 is a side cross-sectional view of a user-interface with an angled baffle with a minimal cross-sectional interface.

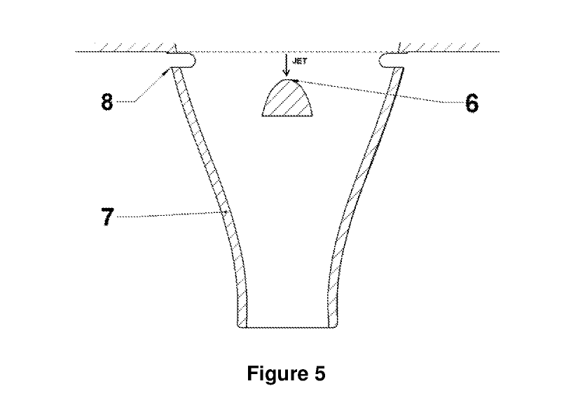

FIG. 5 is a side cross-sectional view of a user-interface with a rounded baffle.

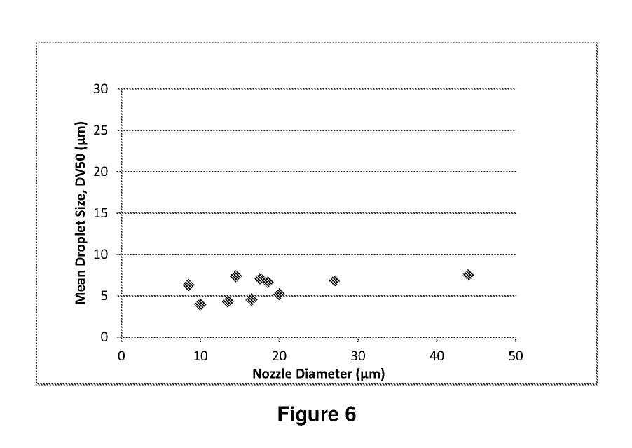

FIG. 6 shows experimental measurements of the mean droplet sizes generated using this method using a pressure of 96 bar, for a range of different outlet hole sizes.

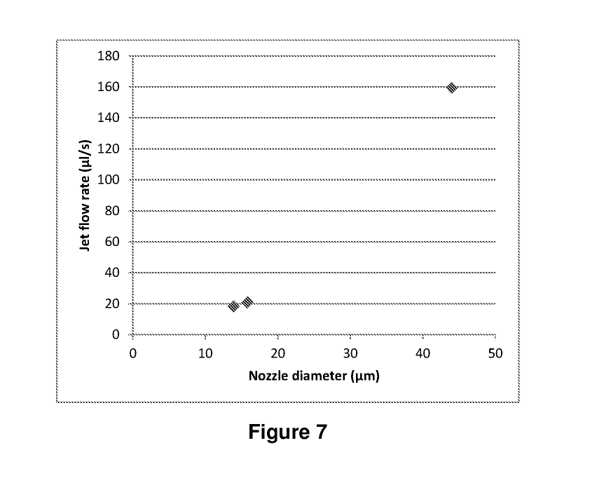

FIG. 7 shows experimental measurements of flow rates through the nozzle with several different outlet hole sizes.

FIG. 1 shows a simple implementation of the present invention. A small volume (approximately 50 μl) of liquid drug or similar solution (1) is contained within a dosing chamber or pressure vessel (2). A piston (3) is used to force the liquid through a mesh (4) containing one or more holes (5) with a diameter of 100 μm or less, at pressures on the order of 100 bar. The liquid forms a fluid jet with a velocity on the order of 100 m/s, with a diameter approximately related to that of the hole in the mesh. An impaction surface or baffle (6) is located approximately 10 mm downstream of the nozzle. The fluid jet collides with the impaction surface and breaks up into droplets, forming a droplet plume with an initial velocity related to the collision angle of the jet with the impaction surface.

The impaction surface can be housed in a component external to the nozzle, including a user interface such as a mouthpiece or nose piece (7). The impaction surface may be moulded as part of the user interface or it may be a separate component. When the fluid jet enters the user interface, it imparts momentum to the surrounding air. The user interface may contain air inlets (8) upstream of the impaction surface such that a stream of air is created within the user interface. The air will entrain droplets in the flow and contribute to the plumes forward momentum out of the user interface. Airflow may also be provided by the user drawing air from the user interface.

In this present embodiment, the mesh is manufactured by laser drilling and consists of a simple straight through hole. Holes with tapered or bell-shaped cross-sections have also been investigated that have smaller inlet pressure losses. Metal or plastic perforate meshes with hole diameters as small as 2 μm can be manufactured at very low cost in high volumes by laser drilling with an excimer laser. A number of other manufacturing routes are also viable, including electroforming and etching. Holes with diameters as small as 30 μm can be formed through injection moulding.

Through this method, a plume of droplets will be generated until the piston reaches the end of its travel and the fluid jet has ceased. After this, the piston can be retracted. The piston may contain a non-return valve (9) such that that fluid will enter the dosing chamber from a reservoir (not shown) when the piston is retracting, refilling the dosing chamber.

FIG. 2 shows an alternate user interface design with a diverging profile. The air streams from the air inlets to the user interface outlet converge upstream of the impaction surface, entraining many of the droplets generated by the impact in the outward airflow. Furthermore, the air streams will diverge as they reach the outlet of the user interface, further slowing the plume down. User interfaces with converging profiles or with cross-flows may also be used to ensure that aerosolised droplets are entrained in the plume and to further engineer the shape and velocity of the resulting plume. The position of the baffle within the user interface is also crucial.

FIGS. 3, 4 and 5 shows a series of impaction surfaces suspended across a user interface by a rod perpendicular to the plane of the page. The design of the impaction surfaces affects the resulting velocity and shape of the plume, both by determining the collision angle of the jet relative to the impaction surface, and by providing resistance to the airflow passing around the baffle. The reduced outlet area also likely increases the velocity of the outward plume.

The first impaction surface, a flat baffle, is shown in FIG. 3. It absorbs the majority of fluid jet's kinetic energy on impact as the surface is perpendicular to the jet. In addition the baffle provides significant resistance to the airflow surround the jet. The coefficient of drag of a flat baffle is typically on the order of 1, indicating that the majority of the air stream is brought to rest. The resulting droplet plume has a very small velocity out of the user interface (on the order of 0.3 m/s), which is a reduction of over 99.5% of the initial velocity of the jet. The airflow resistance that the flat baffle presents could potentially be reduced by minimising its cross sectional area relative to the size user interface (i.e. if the baffle width was less than 1% of the user interface diameter). However the impaction surface must still be large enough to ensure that small fluid jet(s) impact it even with manufacturing tolerances and hence should be at least 2-3 times the jet diameter.

A baffle with an angled shape and a baffle with a rounded shape are shown in FIGS. 4 and 5. When the 100 m/s fluid jet collides with the angled baffle the resulting droplets retain some forward velocity (>2 m/s) out of the user interface due to the oblique collision angle. In contrast, the velocity of droplets after collision with the rounded baffle is less; the surface of the rounded baffle at the point of impact is almost perpendicular to the jet. Regardless, both baffles present significantly less resistance to the airflow around the baffle than the flat baffle (coefficient of drag≈0.5) and the velocities of the resulting droplet plume are larger than that of the flat baffle.

The shape of the impaction surface can also affect the amount of liquid that is deposited on the surface. If the baffle is very large relative to the jet diameter, fluid that does not aerosolise may build up on the baffle. If the surface has sharp corners such as that of the angled baffle (FIG. 4), then fluid that does not aerosolise may run off the surface. The impaction surface may be constructed or coated with non-wetting materials, such as hydrophobic or super-hydrophobic materials to further assist with fluid run-off. A super-hydrophobic coating could be applied onto a moulded plastic baffle that has a desired shape. Remaining solution that has not aerosolised after impact will then bead up on these surfaces and roll off rather than spreading. Another possibility is that the impaction surface may be porous or contain or consist of capillaries to draw fluid away from the site of impact.

FIGS. 6 and 7 present experimental data from one embodiment of the present invention. The results are included as an example and should not be construed as a limit to the capabilities of the invention. FIG. 6 shows the mean droplet sizes (DV₅₀) that are produced using this embodiment at a constant pressure (96 bar). The mean droplet size of the generated plume appears to be largely independent of the hole size of the mesh, and instead depends primarily on the applied pressure. Further experiments (not shown) have demonstrated that much larger droplets (DV₅₀: 15-20 μm) can be produced at lower pressures and with more holes. FIG. 7 shows the flow rate of liquid through the nozzle across a range of hole sizes. These initial experiments indicate that the plume droplet size and flow rate can be tuned independently by appropriate selection of the applied pressure, hole size, and number of holes.

This is likely a consequence of the jet velocity depending almost solely on the applied fluid pressure and not on hole size in the present embodiment. Although the holes are very small, the fluid velocities are very high—the pressure losses due to viscous effects are not dominant (<10%) compared to the pressure accelerating the fluid. The velocity of the fluid is almost solely a function of the pressure applied to the fluid and its density

$$( V \approx \sqrt{\frac{2 p}{ρ}} ) .$$

The flow rate of liquid through the hole is a function of the velocity of the jet multiplied by the hole area.

The droplet sizes generated by the collision are likely to be a strong function of the jet velocity and only a weak function of the jet diameter.

There are a number of low cost portable drive mechanisms that can be used to power the invention at the required pressures, due to the low volumes of liquid being expelled. The energy required to expel the fluid is modest; only 500 mJ is required to expel a 50 μl dose under a pressure of 100 bar. The user could prime an energy storage mechanism such as a coil spring or air spring and then trigger it later to expel the dose. The spring would only need to be compressed with a force of 30 N so it can apply a pressure of 100 bar to a 2 mm diameter piston. If the spring free length is much longer than the 16 mm piston travel, i.e. 150 mm, and the spring rate is small (0.3 N/mm), than the applied force will be nearly constant for the duration of firing. The spring could be pre-compressed such that the user only needs to apply the 30 N over the 16 mm travel distance. Even without mechanical advantage, a typical user could apply this force with their hands. There are many other alternative drive sources, including a compressed gas source such as a canister of CO₂. The vapour pressure of liquid CO₂ at room temperature is 65 bar and a valve could be used to vent CO₂ from the canister onto the piston, or directly onto the drug.

1. A spray device for generating an aerosol, the device comprising;
a perforate element comprising one or more nozzles, each nozzle having an inlet and an outlet and having a diameter of no more than 100 μm;
a drive mechanism for causing, in use, a liquid to be driven through the one or more nozzles, thereby forming a liquid spray having one or more streams of the liquid; and
at least one impaction surface onto which, in use, the liquid impacts, the impaction surface being located downstream of the one or more nozzle outlet.

2. A device according to claim 1, wherein the aerosol is a liquid medicament of any of a liquid drug, solution, suspension and colloid.

3. A device according to claim 1, wherein the perforate element is a laser drilled mesh.

4. A device according to claim 1, wherein the perforate element is an electro formed mesh.

5. A device according to claim 1, wherein the perforate element is a molded structure.

6. A device according to claim 1, wherein the perforate element is a mesh having at least one etched hole therethrough.

7. A device according to claim 1, wherein the diameter of each nozzle no more than 70 μm.

8. A device according to claim 1, wherein the diameter of each nozzle is no more than 30 μm.

9. A device according to claim 1, wherein the impaction surface is located on a baffle downstream of the one or more nozzle outlet.

10. A device according to claim 9, wherein the baffle includes a flat plate perpendicular to a direction of flow through the perforate element, such that the one or more streams of liquid impact the impaction surface perpendicularly.

11. A device according to claim 1, wherein the impaction surface includes, in part or wholly, an angled or curved surface.

12. A device according to claim 1, wherein the impaction surface is formed on a wire, pin or bladed structure having a width at least twice the width of the liquid spray.

13. A device according to claim 1, wherein the impaction surface includes one or more capillary tubes or wicks that convey the liquid away from the impaction surface by capillary action.

14. A device according to claim 1, wherein the impaction surface includes a porous material such that the liquid deposited adjacent to the impaction surface is wicked away by the porous material.

15. A device according to claim 1, wherein the impaction surface includes a hydrophobic material such that the hydrophobic material reduces the retention of liquid droplets on the impaction surface.

16. A device according to claim 1, wherein the impaction surface is spaced away from the nozzle outlet by at least 1 mm.

17. A device according to claim 16, wherein the impaction surface is spaced away from the nozzle out-let by between 10 mm and 35 mm.

18. A device according to claim 1, wherein the impaction surface is located within a user interface.

19. A device according to claim 18, wherein the user interface is any of a mouthpiece and a nosepiece.

20. A device according to claim 18, wherein the user interface is a separably fixed to the device.

21. A device according to claim 18, wherein the user interface is integrally formed with the device.

22. A device according to claim 18, wherein the impaction surface is formed by an internal surface of a wall of the user interface.

23. A device according to claim 22, wherein the impaction surface forms part of a spray pathway from the nozzles to an outlet of the user interface.

24. A device according to claim 1, further comprising a fluid chamber located in fluid communication with the inlet side of the one or more nozzles and which, in use, contains the liquid to be dispensed.

25. A device according to claim 1, wherein the drive mechanism includes any of a piston and a plunger for causing the liquid to be expelled through any one or all of the one or more nozzles.

26. A device according to claim 25, further comprising a biasing element for causing any of the piston and the plunger to move within a fluid chamber to expel the fluid through any one or all of the one or more nozzles, the fluid chamber being located in fluid communication with the inlet side of the one or more nozzles and which, in use, contains the liquid to be dispensed.

27. A device according to claim 26, further comprising an actuator for retracting any of the piston and plunger to compress the biasing element.

28. A device according to claim 26, further comprising an actuator for compressing the biasing element, such that the plunger can then be retracted.

29. A device according claim 24, further comprising a oneway valve within the fluid chamber.

30. A device according to claim 18, further comprising one or more air inlets within the user interface.

31. A device according to claim 30, wherein the air inlets are located on the upstream side of the impaction surface.

32. A device according to claim 1, wherein the device is any of a nebulizer and an inhaler.

33. A device according to claim 18, wherein the user interface is suitable for use with any of an oral, nasal and ophthalmic use.

34. A device according to claim 1, wherein the outlet includes a first set of one or more holes, each hole defining a first dimension, the device further comprising a second perforate element having a second set of holes, each of the second set of holes defining a second dimension, the second dimension being smaller than the first dimension, the second set of holes and having a larger number of holes than the first set of holes and with the second perforate element being arranged to act as a filter.

35. A device according to claim 34, wherein the second perforate element is formed from a laser-drilled mesh.

36. A method of generating an aerosol comprising the steps of:
providing a liquid to an inlet side of a perforate element having one or more nozzles having a diameter of no more than 100 μm;
driving the liquid through the perforate element to create a liquid spray having one or more streams of the liquid; and
impacting the liquid spray onto an impaction surface located downstream of the nozzle.

37. A method of generating an aerosol of a liquid medicament of any of a liquid drug, solution, suspension and colloid, the method comprising the steps of:
providing a liquid to an inlet side of a perforate element having one or more nozzles having a diameter of no more than 100 μm;
driving the liquid through the perforate element to create a liquid spray having one or more streams of the liquid; and
impacting the liquid spray onto an impaction surface located downstream of the nozzle to create an aerosol.

38. A method according to 36, wherein the method uses a device according to claim 1.

39. A method according to claim 36, wherein a pressure applied to drive the liquid through the perforate element is greater than 10 bar.

40. A method according to claim 36, wherein impaction with the impaction surface creates droplets of the liquid having a mean diameter of less than 30 μm.

41. A method according to claim 36, wherein the liquid is driven through one or more nozzles having a diameter of no more than 30 μm.
